(19) United States
(12) Patent Application Publication
(10) Pub. No.: US 2011/0318348 A1
(43) Pub. Date: Dec. 29, 2011

(54) INDUCTION OF MUCOSAL IMMUNE RESPONSES BY MUCOSAL DELIVERY PENTABODY COMPLEX (MDPC)

(75) Inventors: Jianbing Zhang, Ottawa (CA); Matthew J. Henry, Indianapolis, IN (US); Wangxue Chen, Nepean (CA); Steven R. Webb, Westfield, IN (US)

(73) Assignees: National Research Council of Canada, Ottawa, ON (CA); Dow AgroSciences LLC, Indianapolis, IN (US)

(21) Appl. No.: 13/124,062

(22) PCT Filed: Oct. 13, 2009
(86) PCT No.: PCT/US09/60495

§ 371 Date: Jul. 5, 2011

Related U.S. Application Data
(60) Provisional application No. 61/105,305, filed on Oct. 14, 2008.

(51) Int. Cl. A61K 39/395; A61P 31/00; A61P 37/08; A61P 37/04

(52) U.S. Cl. 424/135.1

(57) ABSTRACT

The subject invention provides, for example, a novel approach to specifically induce intranasal and/or oral mucosal as well as humoral antibody response by administrating a mucosal delivery pentabody complex (MDPC). The MDPC is a complex formed by mixing a target antigen and a mucosal delivery pentabody (MDP) that has a strong affinity to the target antigen. The MDP is a fusion protein of a single domain antibody (sdAb; which binds to the target antigen specifically) to a pentamerization domain (which can include the B-subunit of an AB5 toxin family, including the B subunit of cholera toxin (CT) or heat-labile toxin (LT)). The pentamerization domain can self-assemble into a pentamer, through which a pentameric single domain antibody, or a pentabody, is formed.

### BACKGROUND OF THE INVENTION

WO 03/046560 (equivalent to US 20060051292) relates in part to improving binding properties of antibodies. This reference contains no reference to mucosal vaccines.

Despite the early success of mucosal vaccines such as polio vaccine, induction of effective and long-lasting antigen-specific mucosal immune response remains a challenge (1). Since most antigens do not spontaneously induce effective mucosal immunity, adjuvants are typically required to develop effective oral or nasal vaccines.

Currently tested mucosal immunization systems include attenuated mutants of bacteria, different formulations of liposomes encapsulation of antigens into microspheres (2), lipo-structures and virus-like particles (3). Tested mucosal adjuvants include CpG DNA (4), bacterial toxins (5) such as cholera toxin (CT)(6, 7), Escherichia coli heat labile toxin (LT)(8, 9) and their derivatives. Among these, CT- or LT-based adjuvants have been used (10).

Both CT and LT are composed of a B-subunit pentamer, which binds to the cellular receptor G_{M} 1 on nucleated cells (11), and an A subunit monomer, which is an ADP-ribosyltransferase (12) and the toxic entity. Thus, efforts have been made to reduce the toxicity of the toxins by either making non-toxic A subunit mutants or employing only the B-subunit. Separation of adjuvanticity and toxicity of CT or LT was one theory. CT- or LT-variants with preserved adjuvanticity but much reduced toxicity was also hoped for. Although reduction in toxicity in CT and LT is often associated with loss of adjuvanticity, CT variants have been generated (13-17) and are being tested.

Another strategy is to employ the non-toxic, receptor binding B-subunit pentamer of CT or LT, thus being called CTB or LTB. CTB and LTB have been reported to have preserved the adjuvanticity of the holotoxins in some cases. Influenza subunit antigen adjuvanted by LTB was found to induce protective intranasal IgA after i.n. immunization (18). However, addition of CTB or LTB to the antigen did not induce significant mucosal IgA in many reported cases. Fibronectin-binding domain of the SfbI protein of Streptococcus pyogenes was one of them (19).

Yet another approach was to fuse a T-cell and a B-cell epitope of the 28 kDa glutathione-S-transferase of Schistosoma mansoni, to CTB (20).

### BRIEF SUMMARY OF THE INVENTION

The subject invention provides, for example, a novel approach to specifically induce intranasal and/or oral mucosal, as well as humoral, antibody response by administrating a mucosal delivery pentabody complex (MDPC). MDPC is a complex formed by mixing a target antigen and a mucosal delivery pentabody (MDP) that has a strong affinity to the target antigen. The MDP is a fusion protein of a single domain antibody (sdAb; which binds to the target antigen specifically), to a pentamerization domain. In some embodiments, the pentamerization domain can be the B-subunit of an AB5 toxin family (21), particularly the B subunit of cholera toxin (CT) or heat-labile toxin (LT). The pentamerization domain can self-assemble into a pentamer, through which a pentameric single domain antibody, or a pentabody, is formed.

### BRIEF DESCRIPTION OF THE DRAWINGS

FIG. 1 sdAbs and CTB-based pentabodies constructed in this work. (A) Sequences of BSA7, BSA8, BSA12 and BSA16 with the three complimentarity determining regions underlined. (B) Schematic drawing of primary structure of the sdAbs and their pentabodies. The ompA signal peptide will be removed during the secretion of the protein. CTB and the sdAbs are linked by a 15 AA linker (L) and tagged with a c-Myc detection tag (myc) and a 6× histidine purification tag. (C) SDS-PAGE of purified BSA8, BSA12, BSA16, C3C-BSA8, C3C-BSA12 and C3C-BSA16 (lane 1-6).

FIG. 2. Interactions between BSA and the antibodies constructed in this work. (A) Binding of BSA8 to BSA at the concentrations of 10, 20, 40, 60, 80 and 100 nM. (B) Binding of BSA 12 to BSA at the concentrations of 1, 2, 5, 10, 20 and 50 nM. (C) Binding of BSA16 to BSA at the concentrations of 25, 100, 300, 500, 1000, 2000 and 3000 nM. (D) Binding of BSA12 to BSA at 10 nM with extended dissociation time. Open circle are used for real data point and solid line for fitting of 1:1 binding model in B, C and D where the fittings are reasonably good. (E) bindings of 10 nM C3C-BSA8, C3C-BSA12 and C3C-BSA16 to BSA; (F) binding of 10 nM C3C-BSA8 to BSA; (G) binding of 1 μM BSA to C3C-BSA8, C3C-BSA12 and C3C-BSA16 and (H) binding of 1.2 μM BSA to C3C-BSA8. (F) and (H) are to indicate interactions between C3C-BSA8 and BSA, which are invisible in (E) and (G) due to the scales used.

FIG. 3. Formation of protein complexes between the pentabodies and BSA. SEC profiles of BSA (A) and the pentabody C3C-BSA12 (B) on Superdex 200™ are shown at the top panel. Profiles of C3C-BSA8 and C3C-BSA16 are very similar to that of C3C-BSA12 and are not shown. (C) SEC profiles of a mixture of 80 μl 1 mg/ml BSA and 168 μl 1 mg/ml C3C-BSA12 (molar ratio=5:1). Volume of C3C-BSA12 was adjusted to have a molar ratio of 3:1 (D), 2:1 (E) and 1:1 (F). Similarly, SEC profiles of mixtures of C3C-BSA 16 and BSA (G) and C3C-BSA8 and BSA (H) at a molar ratio of 3:1 were shown. All graphs were normalized to 100 to facilitate comparison. C3C-BSA12, for example, is shown to form a tight complex.

FIG. 4. BSA-specific systemic and mucosal immune responses five weeks after the first immunization. Groups of 5 mice were immunized intranasally or orally three times with either PBS, BSA alone (CTRL) or with BSA supplemented with the indicated reagents. On the far right of each panel, BSA-specific immune response after oral BSA/C3C-BSA12 immunization is shown.

FIG. 5. A schematic drawing showing that, to induce secretory IgA (sIgA), formation of a tight complex between the MDP and the target antigen (as in the case of BSA/C3C-BSA12 interaction) is required. Induction of immune response is probably mediated through the binding of pentabody to cellular receptor G_{M} 1 on nucleated cells. Little antigen-specific mucosal immune response was detected when there is no interaction between the target antigen, BSA, and the delivery molecule (such as CTB) or the affinity of the MDP to the target antigen is not strong enough, such as in the case of BSA/C3C-BSA16, to form a tight MDPC. The dissociation constant (K_{D}) of the single domain antibody to the target antigen should be 10⁻⁷ M or lower, preferably 10⁻⁹ M or lower, or most preferably 10⁻¹¹ or lower.

FIG. 6. This figure illustrates a mucosal delivery pentabody complex (MDPC). The MDPC is a complex comprising a target antigen and a mucosal delivery pentabody (MDP). The MDP is a fusion protein of a single domain antibody (sdAb) fused to a pentamerization domain. The sdAb binds to the target antigen specifically and with high affinity. The pentamerization domain can be the B-subunit of an AB₅ toxin family (21).

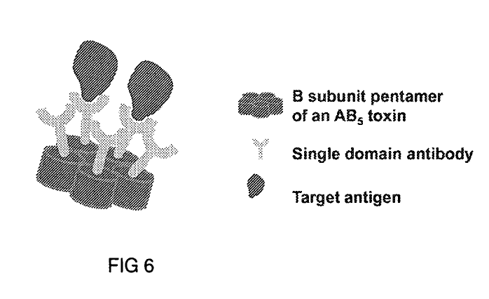

### BRIEF DESCRIPTION OF THE SEQUENCES

SEQ ID NO:1 provides the amino acid sequence of sdAbs “BSA7” as discussed in Example 3, for example.

SEQ ID NO:2 provides the amino acid sequence of sdAbs “BSA8” as discussed in Example 3, for example.

SEQ ID NO:3 provides the amino acid sequence of sdAbs “BSA12” as discussed in Example 3, for example.

SEQ ID NO:4 provides the amino acid sequence of sdAbs “BSA16” as discussed in Example 3, for example.

SEQ ID NO:5 provides the amino acid sequence of lower-affinity pentabody “C3C-BSA8.”

SEQ ID NO:6 provides the amino acid sequence of high-affinity pentabody “C3C-BSA12.”

SEQ ID NO:7 provides the amino acid sequence of lower-affinity pentabody “C3C-BSA16.”

### DETAILED DISCLOSURE OF THE INVENTION

The subject invention provides, for example, specific intranasal and/or oral mucosal delivery of target antigens using mucosal delivery pentabodies to induce mucosal immune response as well as humoral antibody response. This invention also relates in part to a novel procedure of inducing specific mucosal immune response by delivering antigens with pentabodies.

Pentabodies refers to pentameric single domain antibodies (sdAbs). sdAbs refer to variable regions of heavy chains (V_{H}) or light chains (V_{L}) of immunoglobulins. sdAbs from conventional IgGs tend to aggregate because of the hydrophobic portions of the molecular surface of V_{H} or V_{L} which are used to pair with their V_{L} and V_{H} counterparts.

Heavy chain antibodies (HCAbs) naturally devoid of light chains were discovered in camelids (22) such as camel, llama, and alpaca, as well as in sharks (23). The variable regions of these heavy-chain-only Ig molecules, or V_{H}Hs, are solely responsible for antigen binding. Unlike sdAbs from conventional IgGs, sdAbs from camelid HCAbs are not required to interact with light chains and the C_{H}1 domains. Accordingly, more hydrophilic residues were seen (Phe37, Glu44, Arg45 and Gly47) where more hydrophobic ones (Val37, Gly44, Glu45 and Tyr47) are usually used in conventional IgGs. As a consequence, camelid sdAbs usually exist as monomeric proteins when expressed alone (24). Camelids sdAbs for use according to the subject invention are typically non-aggregating, highly thermostable, highly detergent resistant, have relatively high proteolytic resistance, and high affinity by isolation from an immune library or by in vitro affinity maturation. By fusing an sdAb to the B-subunit of shiga toxin 1 (stx 1-B), a pentameric sdAb, or a pentabody, was generated.

Shiga toxin 1 and shiga toxin 2 produced by enterohemorrhagic varieties of E. coli including OH157:H7, together with cholera toxin (Vibrio cholerae), heat-labile enterotoxins (LT and LT-11) (Escherichia-coli), pertussis toxin (Bordetella pertussis), are members of the AB₅ toxin family (21). They are classified as such because each toxin consists of a B subunit pentamer, responsible for receptor binding, and an A-subunit monomer—the toxic entity. The pentabody formed a homogenous pentamer, was relatively resistant to trypsin and chemotrypsin digestion, and had a relatively good thermostability (Tₘ=52° C.). It also retained the binding ability of stx1-B to its cellular receptor G_{b}3. Although Stx1B and the B-subunit of cholera toxin (CTB) or E. coli heat labile toxin (LTB) have relatively low sequence identity, the three proteins share striking structural similarity.

The subject invention relates in part to building pentabodies using CTB as the pentamerization domain and utilizing its G_{M} 1 binding for mucosal antigen delivery. One example of how the subject technology can be applied is in the construction of CTB-pentabodies against a target antigen (BSA is chosen as the model antigen in this case) and uses thereof for the induction of antigen-specific mucosal immune response. In some embodiments of mucosal delivery systems described herein, CTB or LTB is used in the pentabody complex. This allows use of CTB-pentabodies or LTB-pentabodies to bridge antigen and mucosal surfaces, thereby eliminating the requirement of additional adjuvant in the vaccine formulation. Thus, the subject invention relates in part to a novel strategy for the induction of mucosal immune responses with the help of cholera toxin B subunit (CTB).

Some embodiments include linking target antigen to Gₘ 1-expressing cells by pentameric single domain antibodies or “pentabodies.” Single domain antibodies (sdAbs) with different affinities against bovine serum albumin (BSA) as an antigen were raised with phage display technology from the antibody repertoire of a llama immunized with BSA. These antibodies were pentamerizied by fusing them to the CTB to generate pentabodies. The ability of the pentabodies in carrying BSA was found to be directly dictated by their affinities, and this had an impact on their ability to induce BSA-specific immune responses in mice. The high-affinity pentabody C3C-BSA12 (SEQ ID NO:6) was able to induce BSA-specific secretory IgA comparable to that mediated by CT, whereas the lower affinity pentabodies C3C-BSA8 (SEQ ID NO:5) and C3C-BSA16 (SEQ ID NO:7), as well as CTB alone, showed less ability to induce IgA production.

Antigen-specific sdAb fusion to CTB or LTB allows for the entire antigen to be carried to the mucosal surface without producing molecular fusions between the antigen and the B or A subunits of the CT or LT toxins. This platform can be used for further applications and for further development of mucosal vaccines. The results reported herein also clearly demonstrate that antigens can be delivered by CTB-pentabodies to induce antigen specific mucosal immune response.

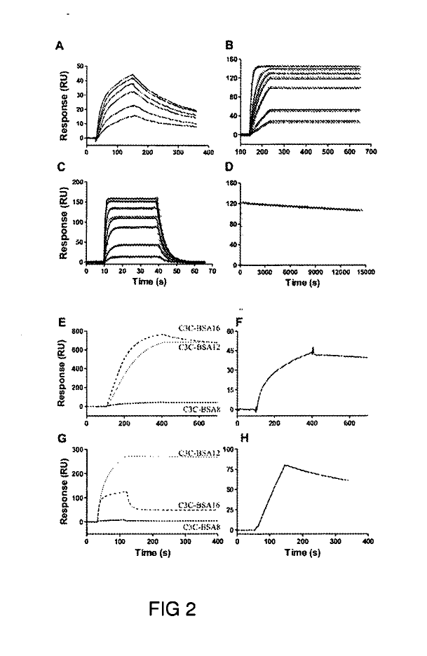

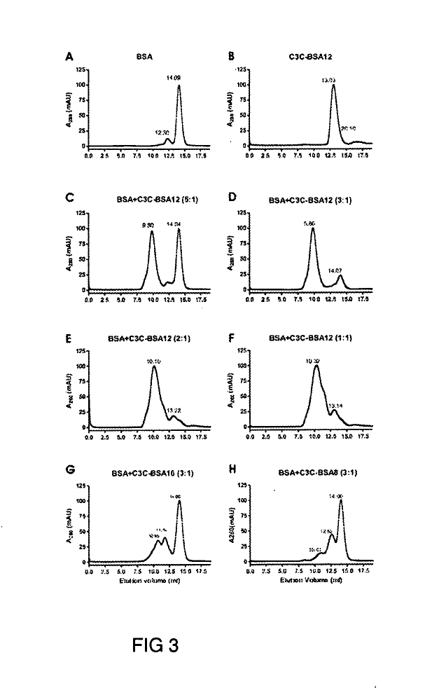

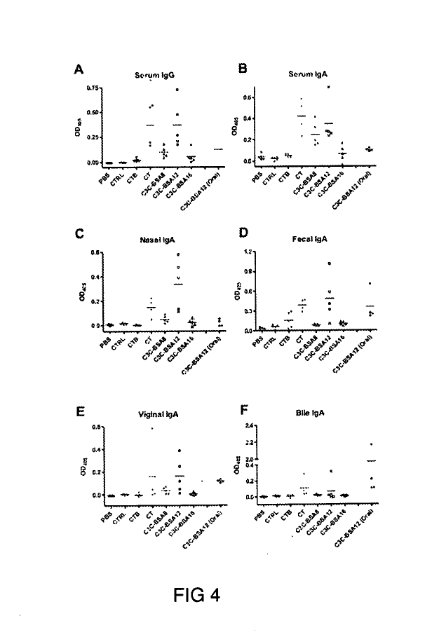

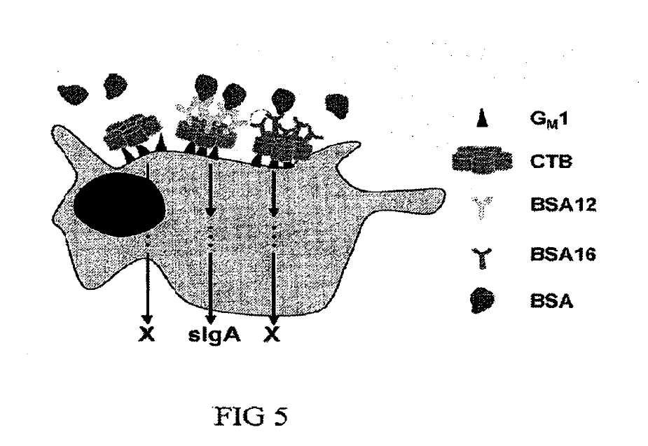

Summarizing some embodiments, presented here is a novel procedure of inducing mucosal immune response, i.e., delivering antigen by a CTB-based pentabody which has strong binding to the antigen. Comparison of biochemical results (FIGS. 2 and 3) and immunological results (FIG. 4) also illustrates the role of CTB in mucosal immunization. In some embodiments, for the purpose of inducing mucosal immune response, the antigen can be tightly associated to the MDP, i.e., a tight complex is formed between both (FIG. 5).

BSA is used and exemplified herein as the antigen. This demonstrates that the subject invention will work with a variety of other antigens. Such other antigens include those obtainable from the following organisms, which are listed (as emerging and re-emerging diseases) on the website for National Institute of Allergy and Infectious Diseases:

### Group I—Pathogens Newly Recognized in the Past Two Decades

### Acanthamebiasis

Australian bat lyssavirus
Babesia, atypical
Bartonella henselae

### Ehrlichiosis

Encephalitozoon cuniculi
Encephalitozoon hellem
Enterocytozoon bieneusi
Helicobacter pylori
Hendra or equine morbilli virus

### Hepatitis C

### Hepatitis E

Human herpesvirus 8
Human herpesvirus 6
Lyme borreliosis

### Parvovirus B19

### Group II—Re-emerging Pathogens

### Enterovirus 71

Clostridium difficile
Mumps virus

### Streptococcus, Group A

Staphylococcus aureus
Group III—Agents with Bioterrorism Potential

### NIAID—Category A

Bacillus anthracia (anthrax)
Clostridium botulinum toxin (botulism)
Yersinia pestis (plague)
Variola major (smallpox) and other related pox viruses
Francisella tularensis (tularemia)
Viral hemorrhagic fevers
   Arenaviruses
      LCM, Junin virus, Machupo virus, Guanarito virus
      Lassa Fever
   Bunyaviruses
      Hantaviruses
      Rift Valley Fever
   Flaviruses
      Dengue
   Filoviruses
      Ebola
      Marburg

### NIAID—Category B

Burkholderia pseudomallei
Coxiella burnetii (Q fever)
Brucella species (brucellosis)
Burkholderia mallei (glanders)
Chlamydia psittaci (Psittacosis)
Ricin toxin (from Ricinus communis)
Epsilon toxin of Clostridium perfringens
Staphylococcus enterotoxin B
Typhus fever (Rickettsia prowazekii)
Food- and waterborne pathogens
   Bacteria
      Diarrheagenic E. coli
      Pathogenic Vibrios
      Shigella species
      Salmonella
      Listeria monocytogenes
      Campylobacter jejuni
      Yersinia enterocolitica)
   Viruses (Caliciviruses, Hepatitis A)
   Protozoa
      Cryptosporidium parvum
      Cyclospora cayatanensis
      Giardia lamblia
      Entamoeba histolytica
      Toxoplasma
   Fungi
      Microsporidia
Additional viral encephalitides
   West Nile virus
   LaCrosse
   California encephalitis
   VEE
   EEE
   WEE
   Japanese Encephalitis virus
   Kyasanur Forest virus

### NIAID—Category C

Emerging infectious disease threats such as Nipah virus and additional hantaviruses. NIAID priority areas:
Tick-borne hemorrhagic fever viruses
   Crimean-Congo Hemorrhagic Fever virus
Tick-borne encephalitis viruses
Yellow fever
Multidrug-resistant TB
Influenza
Other Rickettsias
Rabies
Prions
Chikungunya virus
Severe acute respiratory syndrome-associated coronavirus (SARS-CoV)
Antimicrobial resistance, excluding research on sexually transmitted organisms*
   Research on mechanisms of antimicrobial resistance
   Studies of the emergence and/or spread of antimicrobial resistance genes within pathogen populations
   Studies of the emergence and/or spread of antimicrobial-resistant pathogens in human populations
   Research on therapeutic approaches that target resistance mechanisms
   Modification of existing antimicrobials to overcome emergent resistance
Antimicrobial research, as related to engineered threats and naturally occurring drug-resistant pathogens, focused on development of broad-spectrum antimicrobials
Innate immunity, defined as the study of non-adaptive immune mechanisms that recognize, and respond to, microorganisms, microbial products, and antigens
Coccidioides immitis (added February 2008)
Coccidioides posadasii (added February 2008)

Examples of pathogens of particular note are underlined above (one each in Group I and Group II, and four pathogens in Group III). These six example pathogens/antigen pairs include bacteria, virus and toxin as well as digestive and respiratory system infections. For these, the antigens/pairs are indicated below in parentheses. Additional such selections and pairings can be made by one skilled in the art having the benefit of the subject disclosure. For each pathogen, more than one antigen can be identified as target antigen.
Hepatitis C (Glycoprotein E1 and E2)
Staphylococcus aureus (PsaA)
Bacillus anthracis (anthrax) (Anthrax toxin)
Diarrheagenic E. coli (Shiga toxin)
Campylobacter jejuni (MOMP)
Influenza (neuraminidase, hemagglutinin)

As the term is used herein, “animal” includes mammals and humans. It also includes cattle, cows, hogs, pigs, horses, chickens, poultry, and production animals. For veterinary applications, the subject invention includes cats, dogs, rabbits, and the like.

All patents, patent applications, provisional applications, and publications referred to or cited herein are incorporated by reference in their entirety to the extent they are not inconsistent with the explicit teachings of this specification.

Unless specifically indicated or implied, the terms “a”, “an”, and “the” signify “at least one” as used herein.

Following are examples that illustrate procedures for practicing the invention. These examples should not be construed as limiting. All percentages are by weight and all solvent mixture proportions are by volume unless otherwise noted.

### EXAMPLES

### Example 1

### Materials

E. coli TG1 and M13KO7 helper phage were purchased from New England Biolabs (Mississauga, Ont.). Expression vector pSJF2H, which expresses 6×His-tagged protein instead of 5×His-tagged protein, as the vector pSJF2(29) does, was kindly provided by Dr. J. Tanha (IBS, NRC). DNA encoding CTB was a gift from Dr. D. Miller (U. of Toronto). CT protein was purchased from Sigma (St. Louis, Mo.) and recombinant CTB from SBL Vaccine AB (Stockholm, Sweden). 5 ml immobilized metal affinity chromatography (IMAC) High-Trap™ chelating affinity column was obtained from GE Healthcare (Uppsala, Sweden).

### Example 2

### Isolation of sdAbs Specific to BSA

A female llama was immunized with BSA. An sdAb phagemid display library was constructed from the V_{H}H repertoire of this llama and this library was used for the isolation of sdAbs against BSA.

The llama immune phage display library was panned against 1 mg/ml BSA that was preadsorbed to a Reacti-Bind™ maleic anhydride activated microtiter plate well. About 10¹¹ phage were added to the well and incubated at 37° C. for 2 hr for antigen binding. After disposal of unattached phage, the wells were washed six times with phosphate buffered saline supplemented with 0.05% Tween 20 (PBST) for round one and washes were increased by one for each additional round. Phage were eluted by 10 min incubation with 100 μl 100 mM triethylamine and the eluate was subsequently neutralized with 200 μl 1M Tris-HCl (pH 7.5). Phage were rescued and amplified using M 13KO7 and used for the next round of panning. After three rounds of panning, eluted phage were used to infect exponentially growing E. coli TG1 and rescued by M13KO7. The produced phage were used in phage ELISA.

For phage ELISA, wells of a 96-well plate were coated overnight with 5 μg/ml BSA and then blocked with 1% casein for 2 hr at 37° C. Phage were preblocked with casein overnight, added to the preblocked wells and incubated for 1 hr. Positive phage clones detected by standard ELISA procedure, which revealed that 35 of the 38 analyzed phage clones bound to BSA.

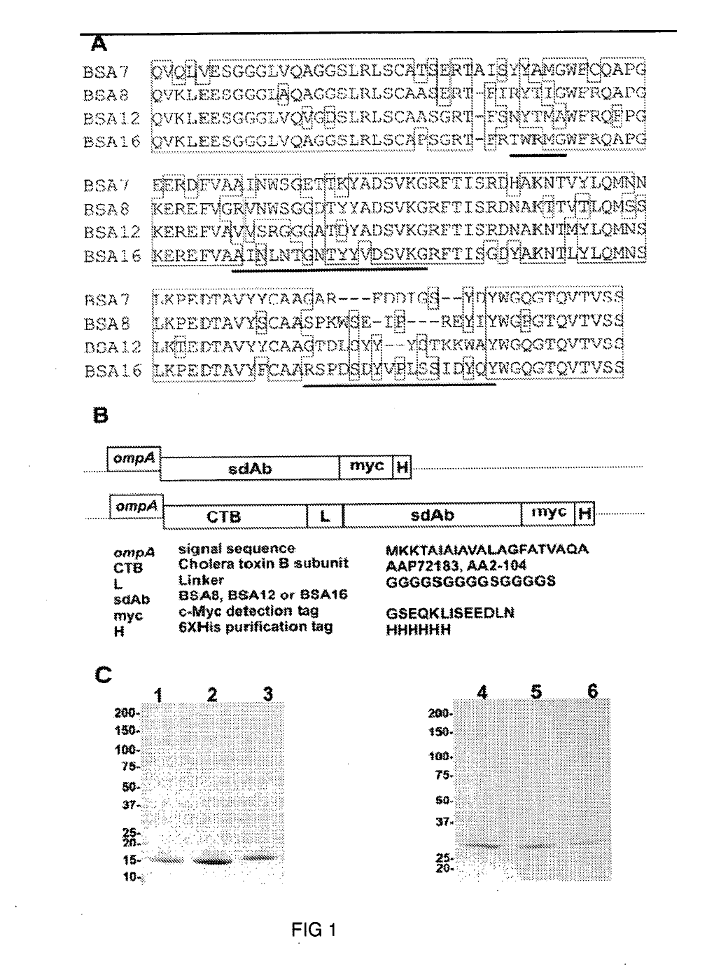

These clones were sent for sequencing. Sequence analysis of these clones revealed four sdAbs: BSA7, BSA8, BSA12 and BSA16 (FIG. 1A).

### Example 3

### Construction, Expression and Characterization of SdAbs

DNA encoding four sdAbs (BSA7, BSA8, BSA12 and BSA16; SEQ ID NOS:1-4, respectively) was amplified by PCR and flanked with BbsI and BamHI restriction sites. The products were cloned into the BbsI and BamHI sites of pSJF2H to generate pBSA7, pBSA8, pBSA12 and pBSA16.

All clones were inoculated in 25 ml LB-Ampicillin (30) and incubated at 37° C. with 200 rpm shaking overnight. The next day, 20 ml of the culture was used to inoculate 1 l of M9 medium (0.2% glucose, 0.6% Na₂HPO₄, 0.3% KH₂PO₄, 0.1% NH₄C1, 0.05% NaCl, 1 mM MgCl₂, 0.1 mM CaCl₂) supplemented with 0.4% casamino acids, 5 mg/l of vitamin BI and 200 μg/ml of ampicillin, and cultured for 24 hr. Next, 100 ml of 10×TB nutrients (12% Tryptone, 24% yeast extract and 4% glycerol), 2 ml of 100 mg/ml Amp and 1 ml of 1 M isopropyl-beta-D-Thiogalactopyranoside (IPTG) were added to the culture and incubation was continued for another 65-70 hr at 28° C. with 200 rpm shaking. E. coli cells were harvested by centrifugation and lysed with lysozyme. Cell lysates were centrifuged, and clear supernatant was loaded onto High-Trap™ chelating affinity columns and His-tagged proteins were purified.

The four sdAb genes were cloned into a periplasmic expression vector pSJF2H to generate sdAb expression vector (FIG. 1B), and the expressed protein was purified by immobilized metal affinity chromatography (IMAC). 3.1, 16.2 and 6.2 milligrams of protein was obtained from one liter of E. coli culture of pBSA8, pBSA12 and pBSA16 (FIG. 1C), respectively. Little protein was obtained from BSA7 expression, and further analysis of this protein was not conducted.

The purified proteins were dialyzed against HBS-E buffer. To assess formation of aggregates or lack thereof, size exclusion chromatography was carried out on BSA8, BSA12 and BSA16 with Superdex 75™ or Superdex 200™ column (Amersham Pharnacia, Piscataway, N.J.) in HBS as described previously (31). The elusion volume of BSA8 (11.87 ml), BSA12 (11.72 ml) and BSA16 (11.80 ml) on a Superdex 75™ column suggested that all three proteins existed as monomers based on elution volumes of molecular weight markers run under the same conditions. No aggregation was observed from any of the three proteins.

### Example 4

### Affinity Measurement

The binding kinetics for the interactions of BSA8, BSA12 and BSA16 to immobilized BSA and other SA was determined by surface plasmon resonance (SPR) using BIACORE 3000 (GE Healthcare). 1700 RUs of BSA (Sigma) were immobilized on research grade CM5-sensorchip (BIACORE). Ethanolamine blocked surface was used as a reference. Immobilizations were carried out at the protein concentrations of 50 μg/ml in 10 mM acetate pH4.5 using amine coupling kit supplied by the manufacturer. Antibodies were passed through Superdex 75 (GE Healthcare) column to separate monomer prior to BIACORE analysis.

In all instances, analyses were carried out at 25° C. in HBS-E buffer (10 mM HEPES, 150 mM NaCl and 3 mM EDTA, pH7.4) supplemented with 0.005% surfactant P20 at a flow rate of 20 ul/min. The surfaces were regenerated with 100 mM HCl (3 seconds). Data were analyzed with BIAevaluation 4.1 software. Affinities of the three sdAbs BSA8, 12 and 16 to BSA were determined by SPR with Biacore 3000. All three sdAbs bound to BSA specifically (FIG. 2). A higher binding capacity (˜150 RU) was achieved by BSA12 (FIG. 2B) and BSA16 (FIG. 2C), whereas only about 40 RU of response was recorded for the binding of BSA8 to BSA (FIG. 2A). BSA8 showed binding to BSA with a dissociation rate (k_{d}) of 5×10⁻³ l/s and an estimated dissociation constant (K_{D}) in the range of 100 nM. An accurate K_{D} could not be determined because the SPR profiles did not fit to a 1:1 binding model (FIG. 2A).

BSA 12 has an extremely tight binding to BSA (FIG. 2B). To measure an accurate affinity value, which requires longer dissociation time for very high affinity binders, dissociations was monitored again for 5 min at multiple concentrations from 1 to 50 nM and 4 hr at 10 nM (FIG. 2D). Each binding was performed three times, and the obtained data were reproducible. These experiments revealed a kₐ of 2.5×10⁶ M⁻¹s⁻¹ and a k_{d} of 1×10⁻⁵ s⁻¹, giving a calculated K_{D} of 4×10⁻¹² M (Table 1). Despite the extremely slow dissociation rate, the interaction fits nicely into a 1:1 binding model (FIG. 2D).

TABLE 1
Affinities of sdAbs to BSA

| | | k_{d} ± SE (1/s) | K_{D} ± SE (M) |
|---|---|---|---|
| | kₐ ± SE (1/Ms) | (dissociation rate | Dissociation |
| | (association rate | constant +/− | constant +/− |
| | constant +/− | standard error) | standard error |
| (units:) | standard error) | (1/second) | (Molar) |
| BSA8 | NA | NA | ~10⁻⁷ |
| BSA 12 | 2.5 × 10⁶ ± | *9 × 10⁻⁶ ± | 4 × 10⁻¹² |
| | 3.2 × 10³ | 3 × 10⁻⁸ | |
| BSA16 | 1.0 × 10⁶ ± | 2.7 × 10⁻¹ ± | 2.8 × 10⁻⁷ ± |
| | 2.2 × 10⁴ | 5.4 × 10⁻³ | 1.1 × 10⁻⁸ |

NA: data not analyzed
*kd was determined separately with 4 h dissociation time.

Thus, in some preferred embodiments, binding affinity (expressed as dissociation rate constant (Molar)) can be above 10⁻⁷, above 2.8×10⁻⁷, and above 4×10⁻¹².

### Example 5

### Construction, Expression And Characterization of Pentabodies

CTB-based pentabodies were constructed by standard molecular cloning procedures. DNA encoding CTB was amplified by PCR and franked with BbsI restriction site and DNA encoding linker sequence GGGGSGGGGSGGGGS at 5′- and 3′-ends, respectively. DNA encoding BSA8, BSA 12 and BSA 16 was amplified by PCR and flanked with DNA encoding the linker sequence GGGGSGGGGSGGGGS and BamHI restriction site at 5′- and 3′-ends, respectively. CTB and the three sdAbs are fused at DNA level by overlap extension PCR. The final PCR product was digested by BbsI and BamHI and ligated into pSJF2 digested with the same enzymes to generate clones pC3C-BSA8, pC3C-BSA12 and pC3C-BSA16 (FIG. 1).

Expression of the three proteins were carried out as described in Example 3. CTB-based pentabodies were constructed by fusing each of the three isolated sdAbs BSA8, BSA12 and BSA 16 to the C-terminus of CTB with a peptide linker GGGGSGGGGSGGGGS. The generated clones C3C-BSA8, C3C-BSA12 and C3C-BSA16 (FIG. 1B) have a subunit molecular weight of 28,257, 28396 and 28,786 Dalton, respectively. The three proteins were expressed in E. coli and purified by IMAC (FIG. 1C). 10, 23 and 7 mgs of proteins were obtained from C3C-BSA8, C3C-BSA12 and C3C-BSA16, respectively.

### Example 6

### Affinities of the Pentabodies to the Target Antigen

SPR analysis was performed to assess the bindings of the three CTB-based pentabodies again. All three proteins showed specific binding to immobilized BSA (FIG. 2). Behaving similar to their monomeric counterparts, C3C-BSA12 and C3C-BSA16 achieved high capacity bindings (˜700 RU, FIG. 2E) but C3C-BSA8 did not (˜45 RU, FIGS. 2E and 2F).

C3C-BSA12 showed the tightest binding to BSA with a k_{d} slower than 10⁻⁵ l/s. This is very close to the k_{d} of BSA12, which is 9×10⁻⁶ l/s. This result showed that for an sdAb with very low k_{d}, pentamerization apparently did not increase its avidity. This is different from pentamerization of low affinity sdAbs, by which a very large gain in functional affinity can be achieved (see e.g. C3C-BSA8 and C3C-BSA16). Due to the multivalent nature of the bindings, an accurate k_{d} of the bindings could not be calculated.

Binding of BSA to immobilized pentabodies (FIGS. 2G and 2H) revealed similar binding pattern as observed for monomeric bindings. However, dissociation rates were not calculated. A small portion of BSA exists in dimer (FIG. 3A).

### Example 7

### Formation of BSA-Pentabody Complex

Formation of BSA-pentabody complex is important for the delivery of antigens through CTB-pentabodies, and this was tested with size exclusion chromatography (SEC). The three pentabodies and BSA were analyzed with SEC on a Superdex 200™ to determine their ability to form pentamer as CTB does. All three pentabodies were eluted at the volume of about 13 ml on a Superdex 200™ column (FIG. 3B, only the profile of C3C-BSA12 was shown as the three proteins have almost identical graphs). Based on molecular marker run under the same conditions, the actual MW of all three proteins were determined as about 220 kDa. Although this number is between 7 to 8 times of their subunit MW, the three proteins are still considered pentamer based on the crystal structure of CTB.

No monomer was observed from all three proteins. The graphs also showed that the proteins form very little aggregations (FIG. 3B).

Monomeric BSA has an MW of 67 kDa, and a CTB-based pentabody has an estimated MW of about 143 kDa. BSA (FIG. 3A) and C3C-BSA12 (FIG. 3B) have a major elution peaks at 14.09 and 13.03 ml when run on a Superdex 200™ column. When C3C-BSA12 and BSA were mixed at a 5:1 molar ratio, i.e., a 1:1 molar ratio when BSA12 and BSA are concerned, a protein complex was formed (FIG. 3C, peak at 9.80 ml) but a large BSA peak was still visible. At a 3:1 pentabody BSA ratio (FIG. 3D), the BSA peak almost completely disappeared. This suggests that one C3C-BSA12 pentabody is able to carry approximately three BSA molecules. Further reduction of pentabody:BSA ratio to 2:1 (FIG. 3E) and 1:1 (FIG. 3F) resulted in a shift of the complex peak from 9.8 to 10.10 and 10.32 ml, respectively, probably caused by competition of BSA binding sites in a pentabody molecule by BSA.

C3C-BSA16 (FIG. 3G) and C3C-BSA8 (FIG. 3H) also form complex with BSA. But the height of the free BSA peaks at 14.08 ml suggests that the majority of the BSA remains unbound. In addition, the position of the protein complex peaks also suggest that association and dissociation between BSA and C3C-BSA16/C3C-BSA8 are constantly occurring. In conclusion, C3C-BSA12 is able to form tight protein complex with BSA whereas C3C-BSA8 and C3C-BSA16 did not.

### Example 8

### Induction of Antigen Specific Mucosal Immune Response

Six to eight-week-old female Balb/c mice were purchased from Charles Rivers Laboratory (St. Constant, Quebec). The animals were housed in the Animal Facility of the Institute for Biological Sciences, National Research Council of Canada, Ottawa in accordance with the recommendations of the Canadian Council on Animal Care Guide to the Care and Use of Experimental Animals. The experimental protocols were approved by the institutional animal care committee.

Groups of mice (n=5) were immunized at day 0, 14 and 21 either orally (0.1 ml) or intranasally (50 μl) with various vaccine formulations. For oral immunization, the vaccine was administered by gavage via an 18-gauge feeding needle. For i.n. immunization, mice were anesthetized by i.p. injection of ketamine and xylazine at 0.1 mg and 0.05 mg/g body weight, in 0.25 ml injectable saline, and the vaccine was administered alternately to the mouse nostrils using a P100 pipetter. 10 μg BSA with or without supplement of 7.5 μg CTB, 7.5 mg pentabody or 1 μg CT was used in i.n. immunization. 100 μg BSA supplemented with 75 μg pentabody was used in oral immunization.

At day 35, samples were collected for immunological assays. Blood was collected either from the tail vein, or by cardiac puncture of euthanized mice, and the sera were separated by centrifugation. For fecal samples, three to four freshly voided pellets were collected into a 1.5 ml micro-tube stored on ice, and vortexed vigorously in 10×(w/v) of extraction buffer (5% fetal bovine serum, 0.02% sodium azide in PBS). The tube was then centrifuged at 16,000×g for 10 min, and the supernatant was collected. Vaginal wash samples were collected by slowly injecting and withdrawing (3-4 times) 50 ml PBS (pH 7.2) into the vagina of conscious mice, using a P100 pipette. Nasal wash and bile samples were collected after euthanizing the mice by CO₂ asphyxiation. To sample bile, the gall bladder was put into a 0.5 ml micro-tube and 0.1 ml of the extraction buffer was added. The gall bladder was “macerated” by cutting with scissors. The tube was vortexed gently, centrifuged (10,000×g for 5 min), and the supernatant collected. For nasal wash samples, a small cut was made in the upper trachea and a lavage tube inserted 0.5-1.0 cm towards the head. The nasal cavity was flushed with 1 ml PBS, and the wash was collected from the anterior opening of the nose. All samples were stored at −20° C. until assay.

BSA-specific IgA and IgG antibodies were measured by indirect ELISA method. Briefly, 96-well flat-bottom Immunolon 2® microplates (Thermo Electron Corporation, Milford, Mass., USA) were coated with 5 μg BSA/well in 100 μl of 0.1 M bicarbonate buffer (pH 9.6), at 4° C. overnight. The coated plates were washed twice and blocked with 2% skim milk in PBS at room temperature for 1 h. Aliquots (100 μl/well) of appropriately diluted samples were added to duplicate wells, and the plates were incubated at room temperature for 3 h. Unless indicated otherwise, the sample dilutions used for the ELISA assays were 1:2 for fecal and nasal wash IgA, 1:20 for vaginal, serum and bile IgA, and 1:2000 for serum IgG. After washing the plates 3 times, alkaline phosphatase-conjugated goat anti-mouse IgA (1:1000) or IgG H+L (1:3000) were added (all from Caltag Laboratories, Burlingame, Calif., USA), and plates incubated for 1 h at room temperature. Color reactions were developed by the addition of p-nitrophenyl phosphate (pNPP) substrates (Kirkegaard and Perry Laboratories, Inc., Gaithersburg, Md., USA), and optical density (OD) was measured at 405 nm after 10-60 min incubation periods, using an automated ELISA plate reader (Model 354, Thermo Labsystems, Helsinki, Finland) and Multiskan Accent® software (Thermo Labsystems).

In this study, BSA was used as the model antigen to immunize Balb/c mice. CT, CTB and CTB-pentabodies were added to BSA prior to immunization to test their ability to induce or enhance BSA-specific immune responses. Although ovalbumin (OVA) is often used as a model antigen in immunological investigations to test various immunization platforms, there is some background immune response against OVA. This is likely due to its low sequence identity with murine SA (15%). Instead, we chose BSA as the model antigen due to the low background immunity it induces (70% sequence identity with murine SA) based on a sequence identity analysis.

When immunized with BSA alone, no BSA-specific antibodies were detectable in sera, nasal wash, fecal suspensions, vaginal or bile fluid (FIG. 4). The addition of CTB to BSA also did not induce any detectable antibody responses except for a negligible amount of fecal IgA. By contrast, the addition of CT as adjuvant resulted in BSA-specific serum IgG, serum IgA, intranasal and fecal IgA in all mice and bile and vaginal IgA in some mice, demonstrating that, at least with the BSA model antigen, CT is a potent mucosal adjuvant whereas CTB has no adjuvanticity. Immunization with BSA mixed with the three BSA-binding CTB-pentabodies gave varying results. Two of the three CTB-pentabodies, C3C-BSA8 and C3C-BSA16, had very little impact on BSA-specific antibody titers. However, immunization with BSA mixed with C3C-BSA12 induced BSA-specific IgG and IgA titers in serum that were comparable to those induced by BSA adjuvanted with CT. In addition, C3C-BSA12 added to BSA in fact induced stronger nasal and fecal BSA-specific IgA than CT and BSA and similar vaginal and bile BSA-specific IgA responses (FIG. 4). However, bile mucosal response to BSA was not clear, as only one of the mice in the C3C-BSA12 group and only two mice from the CT group responded. These results clearly demonstrated that linking an antigen to CTB via an sdAb is a viable strategy to induce antigen-specific mucosal immune response. This experiment was repeated once and similar results were observed.

In another experiment, five mice were orally immunized BSA/C3C-BSA12 complex. This immunization resulted in mucosal IgA production on digestive and vaginal surfaces as well as in bile. However, very low, if any, IgA response was observed in the nasal wash. Thus, oral immunization does not always induce intranasal sIgA. This observation differs from the consensus in the literature as summarized by Holmgren and Czerkinsky (1).

These results also indicate that the ability of the pentabodies in antigen delivery is dependent on the affinities of the sdAbs: C3C-BSA12 was the only pentabody capable of delivering BSA to induce BSA-specific mucosal immune responses (FIG. 4), and it is also the stronger binding among the three pendabodies. The accurate affinity of the three sdAbs remain determined.

### Example 9

### Further Application

The ability of the fusion protein to form pentabodies has been assessed. Another application of this technology would be in the formation of the antigen—pentabody in a complex plant-cell, bacteria, yeast, or mammalian cell extract which contains the antigen of interest. The antigen-pentabody complex could then be purified from the matrix using an affinity matrix which would bind to the B subunit of the pentabody. The result would be a rapid capture, concentration and formulation of the antigen from the production matrix.

### REFERENCES

1. Holmgren, J., and Czerkinsky, C. (2005) Mucosal immunity and vaccines Nat. Med. 11, S45-53.
2. Sakaue, G., Hiroi, T., Nakagawa, Y., Someya, K., Iwatani, K., Sawa, Y., Takahashi, H., Honda, M., Kunisawa, J., and Kiyono, H. (2003) HIV mucosal vaccine: nasal immunization with gp160-encapsulated hemagglutinating virus of Japan-liposome induces antigen-specific CTLs and neutralizing antibody responses J Immunol. 170, 495-502.
3. Kang, S. M., Guo, L., Yao, Q., Skountzou, I., and Compans, R. W. (2004) Intranasal immunization with inactivated influenza virus enhances immune responses to coadministered simian-human immunodeficiency virus-like particle antigens J. Virol. 78, 9624-32.
4. McCluskie, M. J., and Davis, H. L. (1999) CpG DNA as mucosal adjuvant Vaccine. 18, 231-7.
5. Freytag, L. C., and Clements, J. D. (1999) in “Defense of mucosal surfaces: pathogenesis, immunity and vaccines.” (Draehenbuhl, J. P., and Neutra, M. R., Eds.), Springer, Berlin, Heidelberg, New York, Barcelona, Hong Kong, London, Milan, Paris, Singapore, Tokyo.
6. Elson, C. 0., and Ealding, W. (1984) Generalized systemic and mucosal immunity in mice after mucosal stimulation with cholera toxin J Immunol. 132, 2736-41.
7. Walsh, E. E. (1993) Mucosal immunization with a subunit respiratory syncytial virus vaccine in mice Vaccine. 11, 1135-8.
8. Clements, J. D., Hartzog, N. M., and Lyon, F. L. (1988) Adjuvant activity of Escherichia coli heat-labile enterotoxin and effect on the induction of oral tolerance in mice to unrelated protein antigens Vaccine. 6, 269-77.
9. Guy, B., Hessler, C., Fourage, S., Haensler, J., Vialon-Lafay, E., Rokbi, B., and Millet, M. J. (1998) Systemic immunization with urease protects mice against Helicobacter pylori infection Vaccine. 16, 850-6.
10. Holmgren, J., Czerkinsky, C., Eriksson, K., and Mharandi, A. (2003) Mucosal immunisation and adjuvants: a brief overview of recent advances and challenges Vaccine. 21 Suppl 2, S89-95.
11. Revesz, T., Greaves, M. F., Capellaro, D., and Murray, R. K. (1976) Differential expression of cell surface binding sites for cholera toxin in acute and chronic leukaemias Br J Haematol. 34, 623-30.
12. Vaughan, M., and Moss, J. (1978) Mechanism of action of choleragen J Supramol Struct. 8, 473-88.
13. Dickinson, B. L., and Clements, J. D. (1995) Dissociation of Escherichia coli heat-labile enterotoxin adjuvanticity from ADP-ribosyltransferase activity Infect Immun. 63, 1617-23.
14. Douce, G., Fontana, M., Pizza, M., Rappuoli, R., and Dougan, G. (1997) Intranasal immunogenicity and adjuvanticity of site-directed mutant derivatives of cholera toxin Infect Immun. 65, 2821-8.
15. Giuliani, M. M., Del Giudice, G., Giannelli, V., Dougan, G., Douce, G., Rappuoli, R., and Pizza, M. (1998) Mucosal adjuvanticity and immunogenicity of LTR72, a novel mutant of Escherichia coli heat-labile enterotoxin with partial knockout of ADP-ribosyltransferase activity J Exp Med. 187, 1123-32.
16. Tsuji, T., Yokochi, T., Kamiya, H., Kawamoto, Y., Miyama, A., and Asano, Y. (1997) Relationship between a low toxicity of the mutant A subunit of enterotoxigenic Escherichia coli enterotoxin and its strong adjuvant action Immunology. 90, 176-82.
17. Yamamoto, S., Takeda, Y., Yamamoto, M., Kurazono, H., Imaoka, K., Yamamoto, M., Fujihashi, K., Noda, M., Kiyono, H., and McGhee, J. R. (1997) Mutants in the ADP-ribosyltransferase cleft of cholera toxin lack diarrheagenicity but retain adjuvanticity J Exp Med. 185, 1203-10.
18. Haan, L., Verweij, W. R., Holtrop, M., Brands, R., van Scharrenburg, G. J., Palache, A. M., Agstcribbe, E., and Wilschut, 1 (2001) Nasal or intramuscular immunization of mice with influenza subunit antigen and the B subunit of Escherichia coli heat-labile toxin induces IgA- or IgG-mediated protective mucosal immunity Vaccine. 19, 2898-907.
19. Schulze, K., Medina, E., Chhatwal, G. S., and Guzman, C. A. (2003) Stimulation of long-lasting protection against Streptococcus pyogenes after intranasal vaccination with non adjuvanted fibronectin-binding domain of the SfbI protein Vaccine. 21, 1958-64.
20. Lebens, M., Sun, J. B., Sadeghi, H., Backstrom, M., Olsson, I., Mielcarek, N., Li, B. L., Capron, A., Czerkinsky, C., and Holmgren, J. (2003) A mucosally administered recombinant fusion protein vaccine against schistosomiasis protecting against immunopathology and infection Vaccine. 21, 514-20.
21. Merritt, E. A., and Hol, W. G. (1995) AB5 toxins Curr Opin Struct Biol. 5, 165-71.
22. Hamers-Casterman, C., Atarhouch, T., Muyldermans, S., Robinson, G., Hamers, C., Songa, E. B., Bendahman, N., and Hamers, R. (1993) Naturally occurring antibodies devoid of light chains Nature. 363, 446-8.
23. Greenberg, A. S., Avila, D., Hughes, M., Hughes, A., McKinney, E. C., and Flajnik, M. F. (1995) A new antigen receptor gene family that undergoes rearrangement and extensive somatic diversification in sharks Nature. 374, 168-73.
24. Zhang, J., Tanha, J., Hirama, T., Khieu, N. H., To, R., Tong-Sevinc, H., Stone, E., Brisson, J. R., and MacKenzie, C. R. (2004) Pentamerization of single-domain antibodies from phage libraries: a novel strategy for the rapid generation of high-avidity antibody reagents J Mol. Biol. 335, 49-56.
25. Dumoulin, M., Conrath, K., Van Meirhaeghe, A., Meersman, F., Heremans, K., Frenken, L. G., Muyldermans, S., Wyns, L., and Matagne, A. (2002) Single-domain antibody fragments with high conformational stability Protein Sci. 11, 500-15.
26. Arbabi Ghahroudi, M., Desmyter, A., Wyns, L., Hamers, R., and Muyldermans, S. (1997) Selection and identification of single domain antibody fragments from camel heavy-chain antibodies FEBS Lett. 414, 521-6.
27. Davies, J., and Riechmann, L. (1996) Affinity improvement of single antibody VH domains: residues in all three hypervariable regions affect antigen binding Immunotechnology. 2, 169-79.
28. De Genst, E., Handelberg, F., Van Meirhaeghe, A., Vynck, S., Loris, R., Wyns, L., and Muyldermans, S. (2004) Chemical basis for the affinity maturation of a camel single domain antibody J Biol. Chem. 279, 53593-601.
29. Tanha, J., Muruganandam, A., and Stanimirovic, D. (2003) Phage display technology for identifying specific antigens on brain endothelial cells Methods Mol. Med. 89, 435-49.
30. Drake, A. W., Myszka, D. G., and Klakamp, S. L. (2004) Characterizing high-affinity antigen/antibody complexes by kinetic- and equilibrium-based methods Anal Biochem. 328, 35-43.
31. Zhang, J., Li, Q., Nguyen, T. D., Tremblay, T. L., Stone, E., To, R., Kelly, J., and Roger MacKenzie, C. (2004) A pentavalent single-domain antibody approach to tumor antigen discovery and the development of novel proteomics reagents J Mol. Biol. 341, 161-9.

We claim:

1. A method for delivering at least one antigen to an animal, said method comprising administering a mucosal delivery pentabody complex (MDPC) to the animal by a mucosal route, said MDPC comprising
a target antigen and
a mucosal delivery pentabody (MDP) that has affinity to the target antigen, wherein
said MDP is a fusion protein comprising
a pentamerization domain and
a single domain antibody (sdAb) fused to said pentamerization domain,
wherein said sdAb specifically binds to the target antigen,
said pentamerization domain comprising the B subunit of an AB5 toxin.

2. The method of claim 1 wherein said B subunit is from a cholera toxin (CT) or a heat-labile toxin (LT).

3. The method of claim 1 wherein said MDP comprises a linker joining said sdAb to said pentamerization domain.

4. The method of claim 1 wherein said MDP self-assembles as a pentamer.

5. The method of claim 1 wherein said sdAb is fused to the N-terminus or the C-terminus of the pentamerization domain.

6. The method of claim 1 wherein the target antigen is a protective antigen.

7. The method of claim 1 wherein the MDP and the target antigen form a complex with high affinity.

8. The method of claim 1 wherein the target antigen is mixed with the MDP, and the MDP mixture is administered to said animal.

9. The method of claim 8 wherein the MDP and the target antigen are mixed at a molar ratio of 1:1 to 1:5.

10. The method of claim 1 wherein said mucosal route is nasal, ocular, oral, rectal, or vaginal.

11. The method of claim 1 wherein said method induces an antigen-specific mucosal immune response.

12. The method of claim 1 wherein said method induces an antigen-specific humoral immune response.

13. The method of claim 1 wherein said method induces an antigen-specific cellular immune response.

14. The method of claim 11 wherein said immune response is characterized by an antigen-specific secretive IgA antibody response at mucosal sites.

15. A mucosal vaccine comprising an MDPC, said MDPC comprising
a target antigen and
a mucosal delivery pentabody (MDP) that has a high affinity to the target antigen, wherein
said MDP is a fusion protein comprising
a pentamerization domain and
a sdAb fused to said pentamerization domain,
wherein said sdAb specifically binds to the target antigen, and
said pentamerization domain comprising the B subunit of an AB5 toxin.


SEQUENCE LISTING (7 sequences)

SEQ ID NO: 1 - protein, 141 aa, Artificial Sequence
  BSA7
QVQLVESGGGLVQAGGSLRLSCATSERTAISYYAMGWFCQAPGEERDFVAAINWSGETTKYADSVKGRFTISRDHAKNTVYLQMNNLKPEDTAVYYCAAGARFDDIGSYDYWGQGTQVTVSSGSEQKLISEEDLNHHHHHH

SEQ ID NO: 2 - protein, 141 aa, Artificial Sequence
  BSA8
QVKLEESGGGLAQAGGSLRLSCAASERTFIRYTIGWFRQAPGKEREFVGRVNWSGGDTYYADSVKGRFTISRDNAKTTVTLQMSSLKPEDTAVYSCAASPKWSEIPREYIYWGPGTQVTVSSGSEQKLISEEDLNHHHHHH

SEQ ID NO: 3 - protein, 142 aa, Artificial Sequence
  BSA12
QVKLEESGGGLVQVGDSLRLSCAASGRTFSNYTMAWFRQFPGKEREFVAVVSRGGGATDYADSVKGRFTISRDNAKNTMYLQMNSLKTDTAVYYCAAGTDLSYYYSTKKWAYWGQGTQVTVSSGSEQKLISEEDLNHHHHHH

SEQ ID NO: 4 - protein, 145 aa, Artificial Sequence
  BSA16
QVKLEESGGGLVQAGGSLRLSCAPSGRTFRTWRMGWFRQAPGKEREFVAAINLNTGNTYYVDSVKGRFTISGDYAKNTLYLQMNSLKPEDTAVYFCAARSPDSDYVPLSSIDYQYWGQGTQVTVSSGSEQKLISEEDLNHHHHHH

SEQ ID NO: 5 - protein, 260 aa, Artificial Sequence
  C3C-BSA8
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMANGGGGSGGGGSGGGGSSGQVKLEESGGGLAQAGGSLRLSCAASERTFIRYTIGWFRQAPGKEREFVGRVNWSGGDTYYADSVKGRFTISRDNAKTTVTLQMSSLKPEDTAVYSCAASPKWSEIPREYIYWGPGTQVTVSSGSEQKLISEEDLNHHHHH

SEQ ID NO: 6 - protein, 260 aa, Artificial Sequence
  C3C-BSA12
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMANGGGGSGGGGSGGGGSSGQVKLEESGGGLVQVGDSLRLSCAARTFSNYTMAWFRQFPGKEREFVAVVSRGGGATDYADSVKGRFTISRDNAKNTMYLQMNSLKTEDTAVYYCAAGTDLSYYYSTKKWAYWGQGTQVTVSSGSEQKLISEEDLNHHHHH

SEQ ID NO: 7 - protein, 264 aa, Artificial Sequence
  C3C-BSA16
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMANGGGGSGGGGSGGGGSSGQVKLEESGGGLVQAGGSLRLSCAPSGRTFRTWRMGWFRQAPGKEREFVAAINLNTGNTYYVDSVKGRFTISGDYAKNTLYLQMNSLKPEDTAVYFCAARSPDSDYVPLSSIDYQYWGQGTQVTVSSGSEQKLISEEDLNHHHHH